(19) United States
(12) Patent Application Publication
(10) Pub. No.: US 2016/0287837 A1
(43) Pub. Date: Oct. 6, 2016

(54) POD FOR SLEEP AND SECURITY

(71) Applicants: Teri Brashier, El Paso, TX (US); Luis M. Ortiz, Albuquerque, NM (US)

(72) Inventors: Teri Brashier, El Paso, TX (US); Luis M. Ortiz, Albuquerque, NM (US)

(21) Appl. No.: 15/092,225

(22) Filed: Apr. 6, 2016

Related U.S. Application Data
(60) Provisional application No. 62/143,582, filed on Apr. 6, 2015.

(51) Int. Cl. A61M 21/02; A61M 21/00; A47C 31/00; A47C 19/02; A47C 21/00; A47C 21/04; H04W 4/00; A62B 31/00

(52) U.S. Cl. CPC A61M 21/02; H04W 4/005; A61M 21/0094; A62B 31/00; A47C 19/025; A47C 21/003; A47C 21/006; A47C 21/042; A47C 31/008; A47C 31/004; A61M 2021/0066; A61M 2021/0027; A61M 2021/0022; A61M 2021/0044

(57) ABSTRACT

A sleep pod can secure a user inside and promote napping. The pod can include biofeedback mechanisms for aiding users in the inducement of sleep for short periods of time and for use as a fatigue management solution. The pod can include a housing including a base to support cushioning and a human being, a hinged lid, closable onto the base to securely include the human being, and a sleep control module supporting at least one of: temperature, security, noise cancellation, sound generation, vibration production mechanisms, lighting, ventilation, and communications. The housing can be made any combination of fiberglass, wood, plastic, metal, and rubber. The sleep control module can support wireless communication with a handheld device associated with the human being, and can support wireless communication with a remote server via a data communication network.

### INVENTION PRIORITY

The present invention is a continuation of Provisional Patent Application No. 62/143,582, filed Apr. 6, 2015, entitled “Pod for Sleep and Security”, which is hereby incorporated by reference in its entirety.

### TECHNICAL FIELD

The present invention is generally related to system and methods supporting napping in secure conditions. More particularly, the present invention relates to a sleep pod including for securing a user and aiding the user in the inducement of sleep and as a fatigue management solution.

### BACKGROUND

Sleep deprivation has become a way of life given rigorous work schedules, active social and family lives, and the growing amount of stimulus from commuting, electronic media and environments encountered throughout a typical day. Sleep deprivation can impact safety, which can be problematic in certain environments. Sleep deprivation affects a person's productivity at work or school. People tend to rely on caffeine or other stimulants to remain alerts. It has been shown that naps can improve a person's productivity and alertness; however, there are limited opportunities for most people to nap at work or school.

What is needed is a solution to reduce fatigue and improve the alertness of individuals while at work, school or other public places away from home. What are needed are systems that provide people with the opportunity to take short naps.

### SUMMARY

It is an objective of the present invention to provide a sleep pod including biofeedback mechanisms for aiding users in the inducement of sleep for short periods of time, and for use as a fatigue management solution.

It is a feature of the present invention to provide a pod including a sleep management module to control conditions including at least one of: air conditioning, temperature control, biofeedback, an alarm system, and adaptable ergonomics.

It is another feature that an alarm system integrated with the pod can includes a vibration mechanism coupled with light and gentle sounds that smoothly wake up the user after a pre-set interval of up to 25 minutes.

It is another feature that the sleep management module include a user-friendly interface enabling users to easily select and manage environmental parameters and security. The user interface can be included within an inner wall of the pod, or the user interface can be provided remotely on a smartphone interface owned by the user and engaged in a secure wireless communication session with the sleep management module during use.

The pod can include cushions adaptable to the shape of the body to better distribute pressure and create a more comfortable sensation. Materials used in the pod can include antimicrobial, moisture-resistant, easy-to-clean surfaces.

It is another feature that the pod and sleep management module include a feedback module that can track and facilitate a user's rest management, compare user rest with qualitative information and metrics that reflects the improvements of the users, and create a activity history for the user for gauging improvements over time.

It is another feature to enable data retrieved from user to be tracked and analyzed for use by industry. For example, the insurance or medical fields may be interested in such data. Discount for pod use could be encourage in exchange for sharing data associated with a user. Data can be monitored, tracked and analyzed and may be of value to many industry segments.

### FIGURES

FIGS. 1A-C is an illustration of a pod.

FIG. 2 is an illustration of modules associated with a pod.

### DETAILED DESCRIPTION

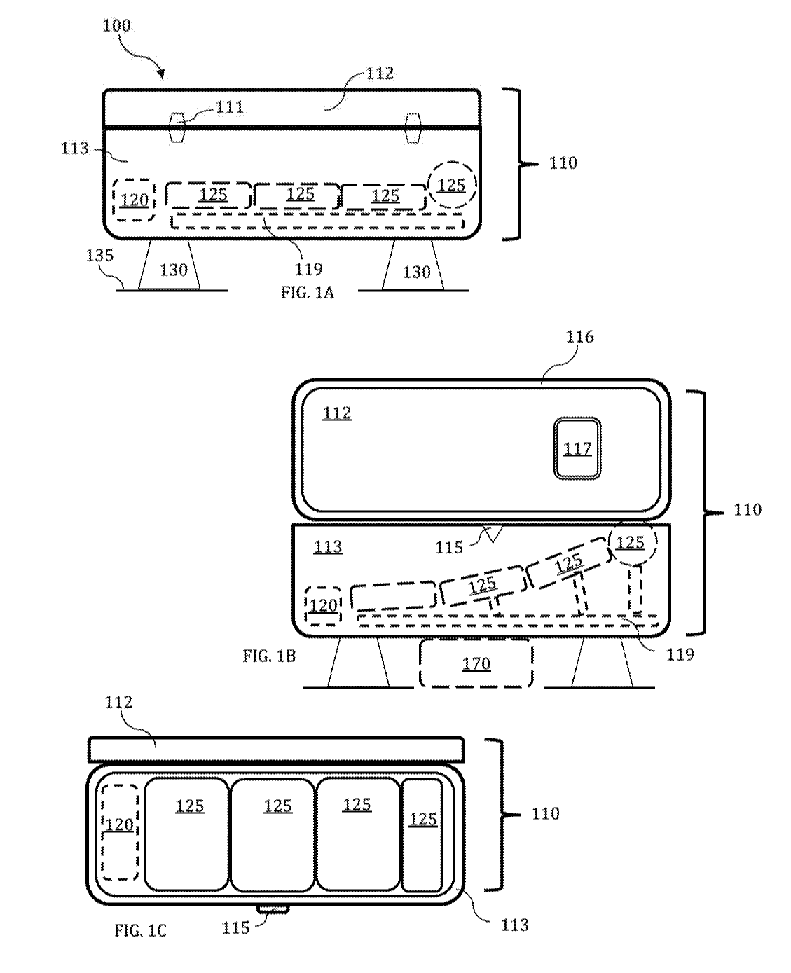

Referring to FIGS. 1A-C, a pod 100 useful for providing personal security and allowing for naps in a public place (e.g., a workplace, school or airport) is illustrated. FIG. 1A is a rear perspective of a pod 100. FIG. 1B is a front perspective of a pod 100. FIG. 1C is a view from a top perspective of a pod 100, with the lid opened. The pod 100 includes an outer Shell, 110, a locking mechanism 115, a sleep control module 120, at least one inner cushion 125, and a foundation 130 that can be secured to the ground 135. The pod 100 can be provided in a clam-like design (similar to a coffin or tanning bed) with a hinged 111 upper lid/door 112 that can be open upward to allow a user to enter into the lower cavity 113 of the pod 100. A complete seal or shield from the outside environment is preferable in order to provide privacy and security to the user. A seal 116 can be integrated along the outer rim of the lid 119. The outer shell of the pod 100 can be constructed from various materials including fiberglass, metal, wood and steel. Fiberglass may be the most cost-effective material given it pervasive use for accessories, such as camper shells and truck lids, manufactured for the auto industry. Electronics 117, including a flat panel display, user interface and lighting can be integrated into the lid 113 for access by a user contained in the pod 100. Electromechanical system 119 can control cushion position as well as vibration and inflation of the cushions.

It should be appreciated that a pod 100 could also be used as a personal security device. For example, a very high-end pod 100 could be made of steel, could be locked to prevent outside intrusion, can include environmental controls, and can enable wireless communications and beacons for distress call purposes.

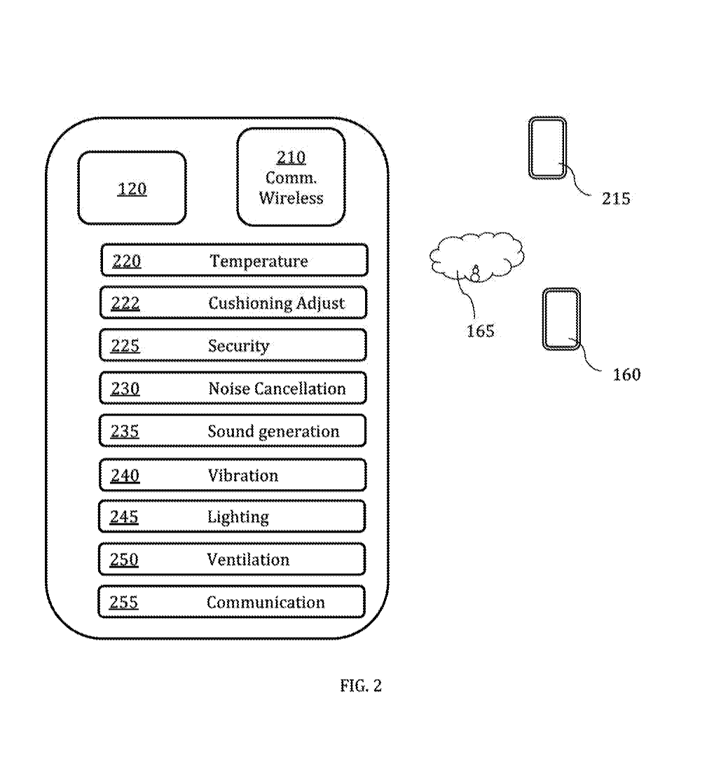

Referring to FIG. 2, as block diagram for modules that can be incorporated into a Pod 100 is illustrated in FIG. 1, is shown. The pod 100 can include a sleep control module 120. The sleep control module can include a wireless interface 210, which can engage in communication with a user's handheld device 215, and can also communicate data with a remote server 160 via a data network 165. The sleep control module can control environmental conditions including at least one of: temperature 220, lighting 212, cushioning 222 (position and inflation), security 225 (e.g., locking of pod and vessel tamper sensors), noise cancellation 230, sound generation 235, vibration production mechanisms 240, lighting 245, ventilation 250, and communications 255 (cellular, WiFi, Bluetooth). Furthermore, an optional storage compartment 170 can be provided to enable users to store their belongings.

1. A pod, comprising:
a housing including a base to support cushioning and a human being;
a hinged lid, closable onto the base to securely include the human being; and
a control module supporting at least one of temperature, security, noise cancellation, sound generation, vibration production mechanisms, lighting, ventilation, and communications.

2. The pod of claim 1, wherein said housing further comprises at least one of fiberglass, wood, plastic, metal, and rubber.

3. The pod of claim 1, wherein said sleep control module supports wireless communication with a handheld device associated with the human being.

4. The pod of claim 1, wherein the sleep control module supports wireless communication with a remote server via a data communication network..

5. A pod, comprising:
a housing including base flooring and cushioning placed on top of the base flowing, said cushioning adapted to support a human being;
a hinged lid, closable onto the base to fully and securely enclose and include the human being; and
a control module supporting at least one of: temperature, security, noise cancellation, sound. generation, vibration production mechanisms, lighting, ventilation, and communications.

6. The pod of claim 5, wherein said housing further comprises at least one of fiberglass, wood, plastic, metal, and rubber.

7. The pod of claim 5, wherein said sleep control module supports wireless communication with a handheld device associated with the human being.

8. The pod of claim 5, wherein the sleep control module supports wireless communication with a remote sewer via a data communication network.

9. A personal security system, comprising:
a housing including base flooring and cushioning placed on top of the base flowing, said cushioning adapted to support a human being;
a hinged lid attached to a side of the housing, where the hinged lid is adapted to be closed onto the base to fully and securely enclose and include the human being inside the housing; and
a control module supporting at least one of temperature, security, noise cancellation, sound generation, vibration production mechanisms, lighting, ventilation, and communications.

10. The pod of claim 9, wherein said housing further comprises at least one of fiberglass, wood, plastic, metal, and rubber.

11. The pod of claim 9, wherein said sleep control module supports wireless communication with a handheld device associated with the human being.

12. The pod of claim 9, wherein the sleep control module supports wireless communication with a remote server via a data communication network.
